(19) United States
(12) Patent Application Publication
(10) Pub. No.: US 2014/0180291 A1
(43) Pub. Date: Jun. 26, 2014

(54) SURGICAL RASP

(71) Applicant: NEURO ENTERPRISES, LLC, Chicago, IL (US)

(72) Inventor: SEAN A. SALEHI, CHICAGO, IL (US)

(73) Assignee: NEURO ENTERPRISES, LLC, CHICAGO, IL (US)

(21) Appl. No.: 14/138,089

(22) Filed: Dec. 22, 2013

Related U.S. Application Data
(60) Provisional application No. 61/797,997, filed on Dec. 21, 2012.

(51) Int. Cl. A61B 17/14

(52) U.S. Cl. CPC A61B 17/14
USPC 606/82

(57) ABSTRACT

In one example, the present invention relates to a surgical instrument for removing tissue from a body, which instrument includes an elongated shaft distally attached to a cutting element, wherein the elongated shaft and the cutting element are fixedly connected by an arcuate element such that the longitudinal axes of the elongated shaft and the cutting element intersect at an angle between about 90° and about 170° and the cutting element comprises a convex non-cutting base that contacts tissue when moving distally. The present invention also relates to a method of using said instrument.

### CROSS REFERENCE TO RELATED APPLICATIONS

This application claims priority to U.S. Provisional Application Ser. No. 61/797,997, filed Dec. 21, 2012, which is incorporated in its entirety herein. This application also claims benefit of U.S. Design Patent Application No. 29,477,449, filed Dec. 22, 2013, which is incorporated in its entirety herein.

### BACKGROUND OF THE INVENTION

The field of the present invention relates generally to surgery involving hard tissue and instruments therefor and, more particularly, to a surgical rasp and method of use thereof

Surgeries involving reduction of bony tissue, often including removal of ligaments or other connective tissues, require use of surgical instruments capable of cutting such tissues but, preferably, are designed such that damage to nontargeted tissue is precluded or minimized. Of particular import is that the surgical instrument design allow the surgeon sufficient control in the process of cutting the hard tissue so that the risk of too much tissue is minimized as well.

One condition suffered by millions of people each year whose cure commonly lends itself to surgery to remove hard tissue pressing upon nerve tissue is associated with pain starting in the lower back and seemingly traveling down the leg is. This symptom is generally known as sciatica. Primary causes of sciatica include a herniated disc or a slipped disc; however, in a significant subset of sciatica patients, the cause is a narrowing of the spinal structure through which the sciatic nerve root exits the spine. That spinal structure is known as the lumbar neuroforamen, which is an opening located between adjacent lumbar vertebrae. The condition by which the sciatic nerve root is compressed at the intervertebral foramen is referred to as a foraminal stenosis.

FIGS. 1A-D present diagrams that depict structures of the spine that play roles in sciatica and its treatment and help orient the description of the utility of the present invention. In particular, the intervertebral foramen 110 is the structure where sciatica pain addressed by the present invention may be relieved by means of increasing space through which the nerve root 109 exits bilaterally from the spine. The nerve root emerges from the spinal canal 107 that encases the spinal cord 108. Providing further orientation to the spinal structure, the spinous process 101, which form the “bumps” of one's backbone, and the vertebral body 106, which faces ventrally, are also shown.

As noted, foraminal stenosis can be the cause of the sciatica by way of compression of sciatic nerve roots emanating from the intervertebral foramen. Foraminal stenosis is commonly treated surgically. For example, a suitable patient may undergo a procedure referred to as a hemilaminectomy and medial facetectomy (“HLMF”). The HLMF refers to two procedures commonly accomplished in concert, namely to remove or reduce the volume of particular structures that are proximal to the compressive intervertebral foramen 110, namely a lamina 104 and a facet joint 112, which is the intersection of the superior and inferior articular facets 102 and 105, respectively. However, removing too much of these structures can result in destabilizing the spine that, in consequence, would require further surgeries, such as a lumbar fusion of adjoining vertebrae with screws and rods. Perhaps needless to say, such further surgeries are accompanied by pain beyond that of the original sciatica symptoms and an extended course of recovery.

A surgical instrument whose design and method of use minimizes the risk of removing too much bony tissue, such as may occur in a HLMF procedure, is needed so that the likelihood of destabilizing the remaining bone structure is minimized.

The present invention presents such a surgical instrument usefully employed in removing hard tissues appropriate, for example, in decompressing the lumbar neuroforamen in the context of a HLMF procedure. In addition, it is contemplated that the surgical instrument of the present invention can be usefully employed in other surgical procedures as well.

### SUMMARY OF THE INVENTION

Provided herein in accordance with exemplary embodiments of the present invention shown in FIGS. 2A-D and FIG. 6 hereof, among other embodiments described herein, is a surgical instrument for removing hard tissue from a body. The instrument comprises, from distal to proximal ends, a cutting element 201, a shaft 202, and a handle 204; the shaft 202 and handle 204 are optionally separated by a transitional element 203 that adapts the thicker diameter of the handle 204 to the narrower diameter of the shaft 202, which shaft itself can taper further as it proceeds distally. The cutting element 201 can be disposed along the longitudinal axis X′, which is indicated in FIG. 2a. Alternatively, the cutting element 201 can be fixedly connected by an arcuate element 207 to the shaft 202 such that the cutting element 201 aligns along a separate longitudinal axis X, as shown in FIG. 2a. In various embodiments of the present invention, the longitudinal axes X′ and X of the elongated shaft and the cutting element, respectively, intersect at an angle between about 90° and about 170°, or between about 120° and about 150° in another set of embodiments. The cutting element 201 as shown in FIG. 2b comprises a convex non-cutting base that contacts tissue when moving distally, i.e., while moving the surgical instrument into the body; a rounded, substantially non-piercing tip 206; and a concave cutting side 205 where, in this embodiment teeth are disposed.

More particularly, (1) the arcuate element 207 has a length substantially that of about a 30° to about a 60° arc of a circle having a radius that is between about 3.5 mm and about 6.5 mm; (2) the surgical instrument further comprises a handle proximally attached to the elongated shaft, either directly or indirectly; if indirectly, the optional transition element is included that is connected to the proximal end of the handle where the transition element substantially matches the diameter and shape of the handle and further connected to the proximal end of the shaft where the transition element substantially matches the diameter and shape of the shaft; (3) the surgical instrument comprises a metal or a polymer that is not toxic when employed in surgery, such as, for example, stainless steel; (4) the cutting element has a curvilinear distal end that is comprised of a convex side that is free of a cutting surface and a concave side that comprises a cutting surface; (5) the cutting element's cross-sectional dimensions relative to its longitudinal axis have a length of between about 0.75 mm and about 5 mm; (6) the surgical instrument, in certain embodiments, further comprises teeth that are disposed on a surface that is opposite the non-cutting base, i.e., on the concave side of the cutting element, wherein angles formed by proximal sides of the teeth and the longitudinal axis X are substantially the same and form an angle between about 20° and about 80°, but in yet other embodiments, the angles formed by proximal sides of the teeth and the longitudinal axis X are not all substantially the same and form an angle between about 50° and about 75°; (7) the surgical instrument can, in yet another embodiment, further comprise a cutting edge disposed at the perimeter of the concave surface, wherein the perimeter of the concave surface and the cutting element contact each other at an angle of between about 90° and about 10°; (8) the cutting element can, in another embodiment, be void of teeth and instead include a concave surface opposite the non-cutting base and a cutting edge disposed at the perimeter of the concave surface, wherein the perimeter of the concave surface and the cutting element contact each other at an angle of between about 90° and about 10°; (9) the length of the cutting element is between about 5 mm and about 18 mm; (10) the elongated shaft has a length range of about 10 cm to about 15 cm; (11) the elongated shaft tapers proximally to distally in its cross-section from about 1 cm to about 1 mm, respectively; (12) the elongated shaft tapers proximally to distally in its cross-section from about 7.5 mm to about 1.5 mm, respectively; and (13) the handle has a length range of about 3 cm to about 5 cm.

In another aspect, the present invention relates to a method for removing targeted tissue from a body employing a surgical instrument that includes a distal cutting element that has a non-cutting convex side and a cutting concave side, comprising (i) making a first incision in the body, (ii) starting with its distal end, passing the surgical instrument from outside the body to a point proximate to the targeted tissue such that predominantly the convex side of the cutting element contacts tissue, (iii) repositioning the surgical instrument so that its concave side of its distal end is in contact with the targeted tissue, (iv) moving the surgical instrument back and forth while applying pressure on the targeted tissue with the concave side of the distal end of the surgical instrument, and (v) removing tissue that is freed from the body.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. Some embodiments of the present invention will now be described with reference to the following description of exemplary embodiments in conjunction with the figures, wherein identical structures, elements or parts that appear in more than one figure are labeled with the same numeral in all of the figures in which they appear, of which:

FIG. 1A is a top (i.e., axial) view of a lumbar vertebra, bony portions only;

FIG. 1B is a side (i.e., lateral) view of a lumbar vertebra, bony portions only;

FIG. 1C is a top (i.e., axial) view of a lumbar vertebra, showing bony portions and spinal nerves;

FIG. 1D is a side view of two lumbar vertebrae, bony portions only;

FIG. 2A is a perspective view of an embodiment of a surgical rasp of the present invention;

FIG. 2B is a side view of the cutting element of one embodiment of a surgical rasp of the present invention;

FIG. 2C is a side view of the enlarged cutting element and arcuate element from FIG. 1A.

FIG. 2D is a side view of the enlarged cutting element and arcuate element showing an alternate tooth design.

FIGS. 3A-F illustrate different embodiments of the surgical rasp of the present invention that utilize various tooth designs for the respective cutting elements thereof;

FIGS. 4A-C illustrate different embodiments of the surgical rasp of the present invention that incorporate a perimeter cutting edge in the respective cutting elements thereof;

FIGS. 5A-F illustrate alternative embodiments of the present invention that replace the teeth with a curette cutting edge.

FIG. 6 illustrates a perspective view of a second embodiment of a surgical rasp of the present invention.

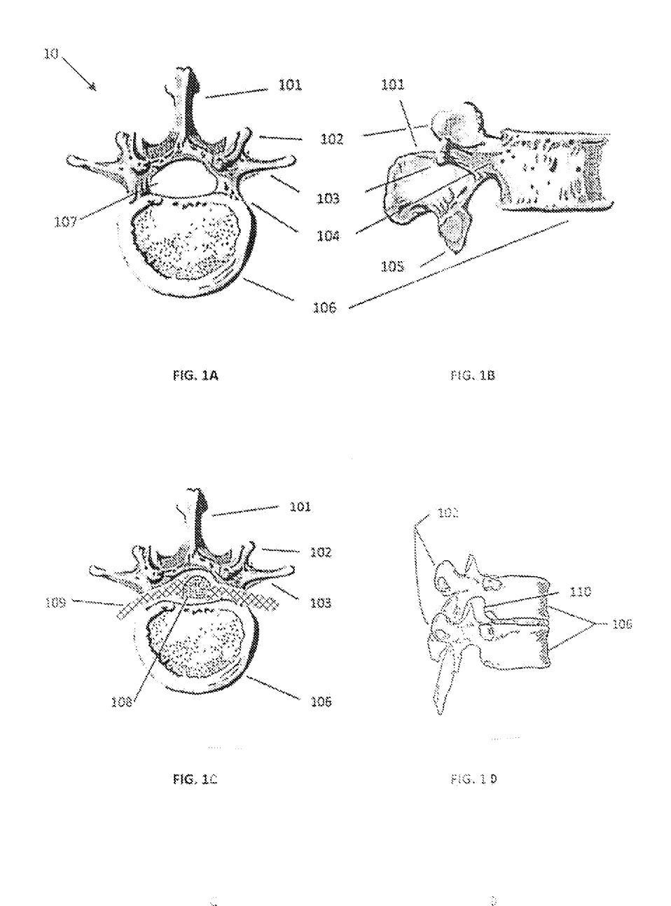

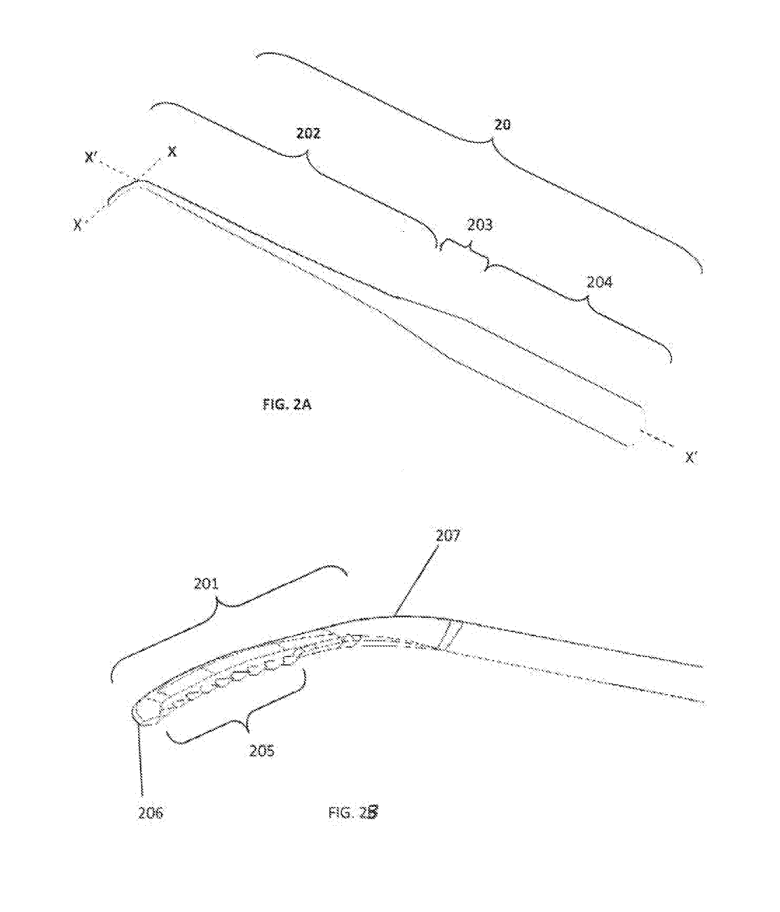

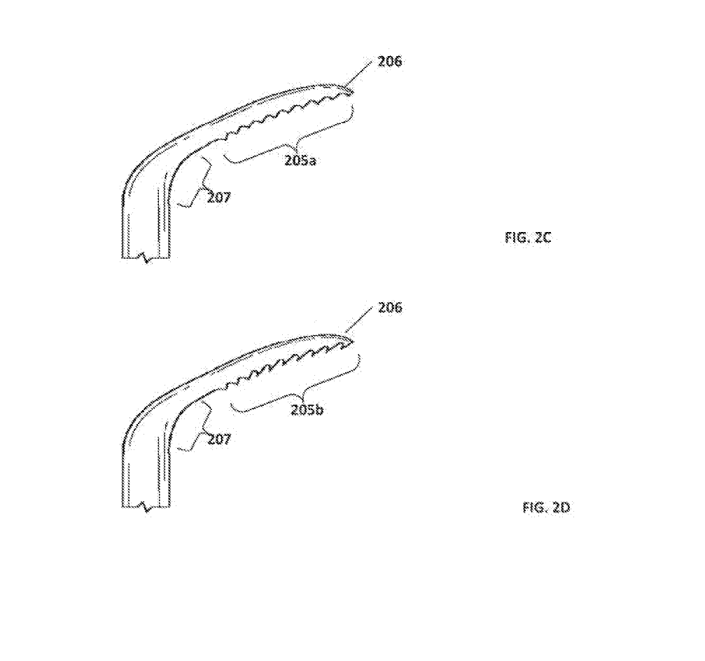

### DETAILED DESCRIPTION OF THE INVENTION

The present invention, in some embodiments thereof, relates to surgical instruments and, more particularly, but not exclusively, to a method for removing tissue from a body that employs said surgical instrument or instruments.

In one example, the present invention relates to a surgical instrument that is usefully employed in a method for decompressing nervous tissue that exits the spine by way of a neuroforamen 110. More particularly, the surgical instrument affords the surgeon a tool for removing bony tissue proximal to the neuroforamen 110, in consequence of which the nervous tissue at and about the neuroforamen becomes decompressed, thereby relieving the symptoms of, for example, sciatica.

The surgical instrument usefully employed for relief of sciatica, for example, is a spinal lumbar foraminal rasp of the present invention. The particular design of the rasp of the present invention, in the hands of a reasonably skilled surgeon, lessens the likelihood of destabilizing the vertebral structure due to removal of more bony tissue than intended. Accordingly, using the inventive rasp lessens the likelihood of requiring further surgeries to correct the unfortunate consequence of destabilizing the vertebral structure. Of course, the surgical instrument may be usefully employed in other procedures for removal of tissue from a patient. The description of the present invention presented herein is exemplary only, and should not limit the range of potential utilities that a skilled artisan may adopt for it.

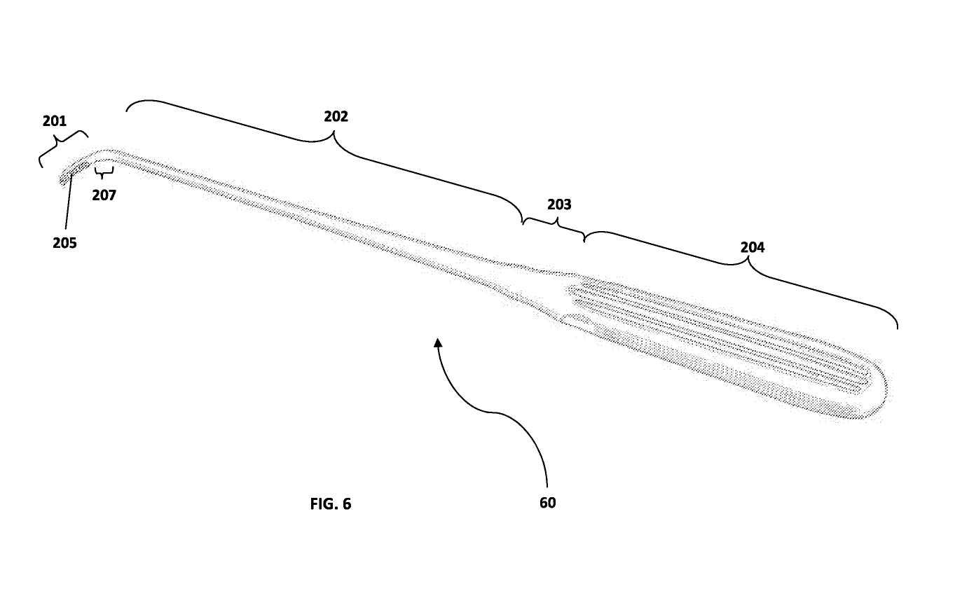

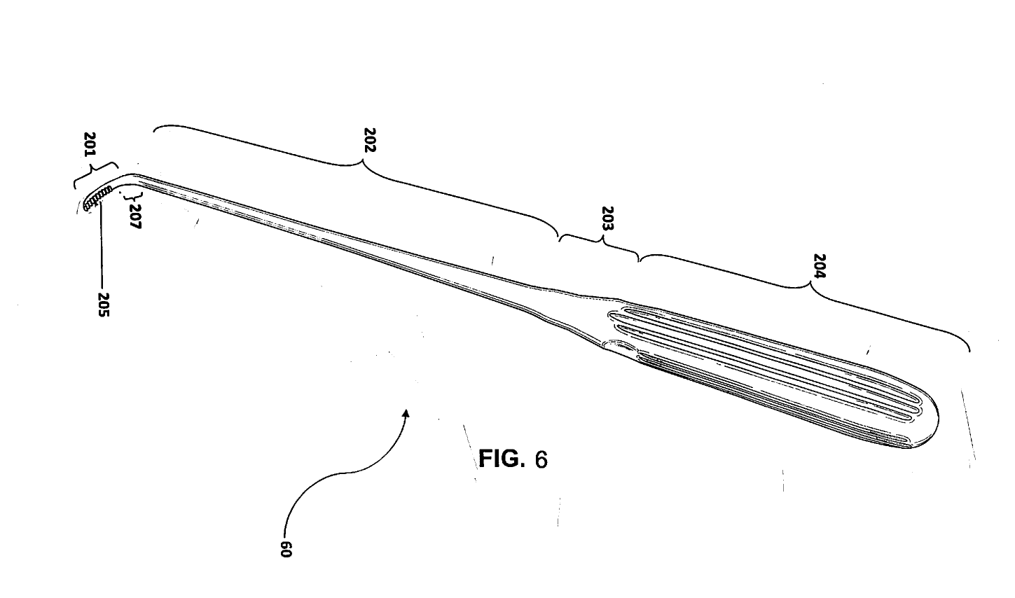

Described herein is a surgical instrument for removing tissue from a body, which tissue includes bone and connective tissues, such as cartilage or ligaments; and further includes calcifications or other hard outcroppings from bone tissue that may occur and cause compression of nervous tissue. As seen in FIGS. 2 and 6, a surgical instrument 20 or 60, referencing now two different embodiments of the present invention, comprises an elongated shaft 202 distally attached to a cutting element 201, wherein the elongated shaft and the cutting element are fixedly attached to an arcuate element 207, such that the longitudinal axes X and X′, respectively, of the cutting element and the elongated shaft intersect at an angle between about 90° and about 170°. In other embodiments, the angle between the noted longitudinal axes is from about 120° and about 150°, about 125° to about 145°, about 130° to about 140°; in yet another embodiment, the angle is about 120°, about 125°, about 130°, about 135° , or about 140°. In an alternative embodiment, the cutting element 201 is substantially collinear with the shaft 202.

The cutting element, in one embodiment as shown in FIG. 2b, includes a curvilinear distal end or tip 206, i.e, the tip of the surgical instrument is substantially not a piercing tip, rather it is a smoothed, rounded tip designed to minimize or prevent damage to tissue as the instrument is inserted into position in the body. The curvilinear distal end of the cutting element includes a gentle curve on its convex side (further described in the next paragraph) for moving past nerve or other tissue without damage to same just as a ski tip tends to slide over outcroppings or other obstacles on a snow trail without substantial impediment to forward motion.

The surgical instrument 20 may be manufactured by any method known and used by artisans in the field of surgical instrument manufacture now or in the future. Without detracting from the utility of the surgical instrument whatsoever, it makes no difference whether it is manufactured as a single piece of material that is cut, ground, burnished, bent, and/or the like into the form of one of the embodiments described herein or assembled from individual parts that conform to the various elements or subgroups of elements of any of the embodiments. Methods used in the manufacture of the surgical rasp of the present invention, as mentioned above, are well-known in the art, of which there are numerous alternative such methods.

The cutting element 201 comprises on one side a curved convex non-cutting base and disposed on the opposite side a concave section that includes, in one embodiment, teeth 205. The convex side contacts tissue when the instrument is inserted into a body, distal end first, and thus protects tissues passed when inserting the instrument from unintended damage. Accordingly, the curved non-cutting base 201 is the convex side of the cutting element 201. As shown in FIG. 2b, the curvature employed for the cutting element 201 is gentle indeed, considering that the arc described by the concave side of the cutting element 201 between the distal end 206 and the arcuate element 207 is part of a circle that has a radius between about 5 cm and 6 cm.

Because the curvature employed for the cutting element of the present invention is so gentle, the concept of a longitudinal axis nonetheless remains viable in defining the angle between the shaft and the cutting element. As shown in FIG. 2A, the longitudinal axis X of the cutting element is the continuation of the line defined by the points signified by the distal tip and distal end of the arcuate element 207 and/or beginning of the teeth 205 of the concave side of the cutting element 201.

The cutting element's cross-sectional dimensions relative to its longitudinal axis X have a length of between about 0.75 mm and about 5 mm; in other embodiments, the cross-sectional dimensions range from about 1 mm to about 4 mm; from about 2 mm to about 3.5 mm; from about 2.5 mm to about 4 mm In other embodiments, the cross-sectional dimension is about 1 mm, about 2 mm, about 3 mm, about 3.5 mm, about 4 mm, about 5 mm, or about 6 mm.

The length of the cutting element, in one embodiment, is between about 5 mm and about 18 mm; in another embodiment, between about 8 mm and about 15 mm; in yet another embodiment, between about 10 mm and about 12 mm; and in yet one other embodiment, the cutting element is about 8 mm, about 10 mm, about 12 mm, about 14 mm, about 16 mm, or about 18 mm in length.

The cutting element 201 of another embodiment is not connected to an arcuate element; instead, in this alternative embodiment (not shown), the cutting element is substantially collinear with the shaft following the longitudinal axis X′.

Those embodiments that include the arcuate element 207, or not, can be manufactured in a process that includes an intermediate form of the instrument 20 that appears to be no different than the instrument just described whose cutting element is substantially collinear with the shaft. The intermediate form can be the final product or, in the instance where the arcuate element is included, the intermediate form would then be subjected to a step of bending the distal end to the angles described herein above. After the bend, the arcuate element 207, as shown in FIG. 2b for another embodiment, has a length substantially that of about a 30° to about a 60° arc of a circle having a radius that is between about 3.5 mm and about 6.5 mm, alternatively between about 4 mm and about 6 mm, between about 4.5 mm and about 5.5 mm, between about 4 mm and about 5 mm; and in other embodiments, the radius is about 3 mm, about 4 mm, about 5 mm, or about 6 mm The arc of the circle, in other embodiments, is about 35°, about 40°, about 45°, about 50°, about 55°; meaning that different embodiments employ different lengths of the arcuate element, generally longer the more obtuse the angle between the longitudinal axes of the cutting element and the shaft.

Upstream of the arcuate element 207 is the shaft 202. In one embodiment, the elongated shaft tapers proximally to distally in its cross-section from about 1 cm to about 1 mm, respectively; in other embodiments, the tapering tends from about 8 mm to about 1.5 mm; from about 7 mm to about 2 mm; from about 7 mm to about 3 mm.

The surgical instrument as shown in FIG. 2A also includes a handle 204 that is proximally attached to the elongated shaft 202. The length of the handle 204 varies in different embodiments of the present invention, but is generally between about 3 cm and about 30 cm; between about 5 cm and about 25 cm; between about 8 cm and about 20 cm; between about 9 cm and about 15 cm; in other embodiments, the length is about 4 cm, about 6 cm, about 8 cm, about 10 cm, about 12 cm, about 14 cm, about 16 cm, about 18 cm, about 20 cm, about 22 cm, about 24 cm, about 26 cm, about 28 cm, or about 30 cm. The width and depth of the handle 204 varies in different embodiments, but generally can be described with respect to a diameter that is between about 1 cm and about 3 cm, between about 1.5 cm and about 2.5 cm, or between about 1.5 cm and about 2 cm; in preferred embodiments, the handle has width-depth dimensions described by a diameter of about 1.5 cm, about 2 cm, about 2.5 cm, or about 3 cm.

The shape of the handle can be any standard shape, such as the substantially square elongate solid displayed in FIG. 2A. Alternative shapes include ovoid elongate or cylindrical solids, as well as cubic or other stubby knobs. In some embodiments, the handle 204 is attached directly to the shaft 202; in other embodiments, the thicker diameter of the handle, generally from about 1 cm to about 2 cm, is adapted to the narrower diameter of the shaft by a transitional element 203, which is an optional element. Generally, the shaft 202 at its proximal end is between about 1 cm and about 5 mm in diameter or between about 9 mm and about 6 mm in diameter,

Overall, the length of the surgical instrument 20 of the present invention ranges from about 15 to cm to about 40 cm; from about 18 cm to about 35 cm; from about 20 cm to about 30 cm; from about 22 cm to about 26 cm; or from about 23 cm to about 25 cm. In other embodiments, the length of the surgical instrument is about 16 cm, 18 cm, about 20 cm, about 22 cm, about 24 cm, about 26 cm, about 28 cm, about 30 cm, about 32 cm, about 34 cm, about 36 cm, about 38 cm, or about 40 cm long.

In some embodiments, the surgical instrument further comprises chisel-like teeth that are disposed on a surface that is opposite the non-cutting base 201a; in certain embodiments, such as that shown in FIG. 2B, the teeth are disposed on a concave surface 204. As a general rule, the surgical instrument 20 is designed to cause minimal or no damage to tissue while being inserted into position in the body. The protective aspects of the invention include (1) the gentle curve of the cutting element where the non-cutting side is convex, thereby sequestering the concave cutting side, the rounded and smooth leading point, and orienting the teeth in a manner that its significantly more effective direction of cutting is the backward motion (i.e., toward the handle), and its significantly less effective direction (for tissue cutting, that is) is forward motion. That difference is heightened by the way the surgeon wields the instrument without any or minimal lateral pressure when inserting, but with measured lateral pressure when pulling the instrument back against target tissue that is desirably removed. The instrument can also be used as a probe to manipulate tissue in view of the design features that minimize damage potential to the tissue just noted.

More particularly, the teeth are inserted or formed in place, using methods well-known in the art, with determined angles between the sides of the teeth and the longitudinal axis of the cutting element that orient the cutting surface of each tooth so that when the instrument is being pulled back, the cutting top edges of the teeth are first contacting tissue, whereas when the instrument is being pushed in, the leading side walls of the teeth buffet tissue, substantially without effect. Of course, if too much lateral force is exerted onto the tissue by the instrument, then the protective design can be defeated, which is why the instrument is for use by a reasonably skilled surgeon when inserted into a body.

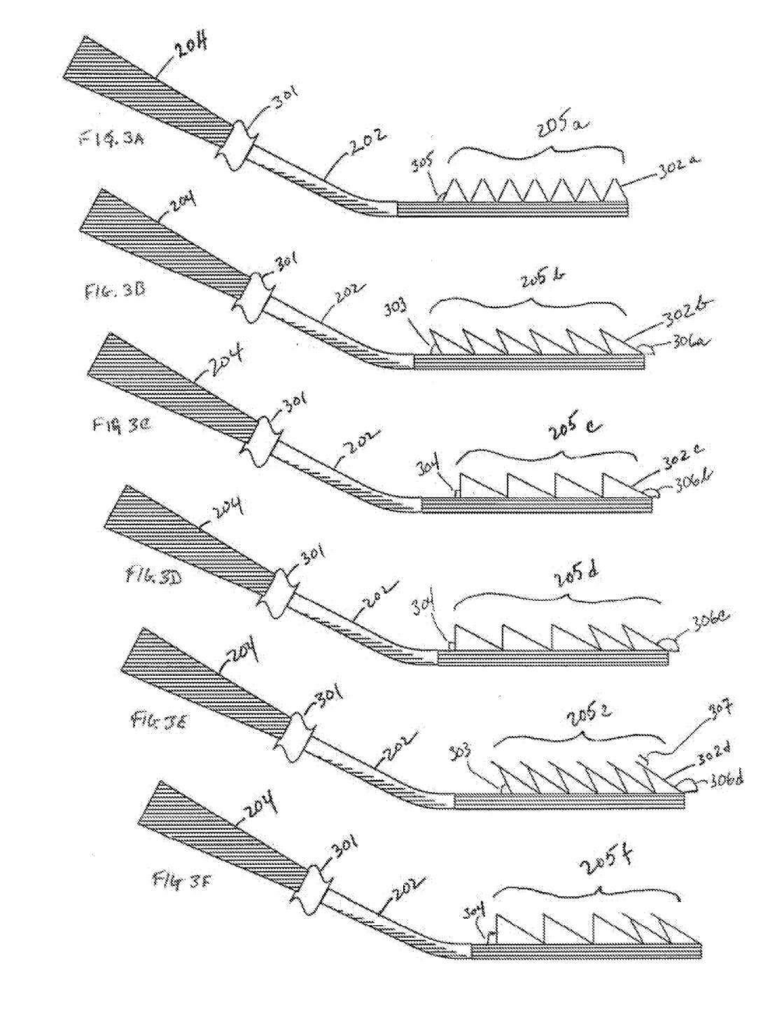

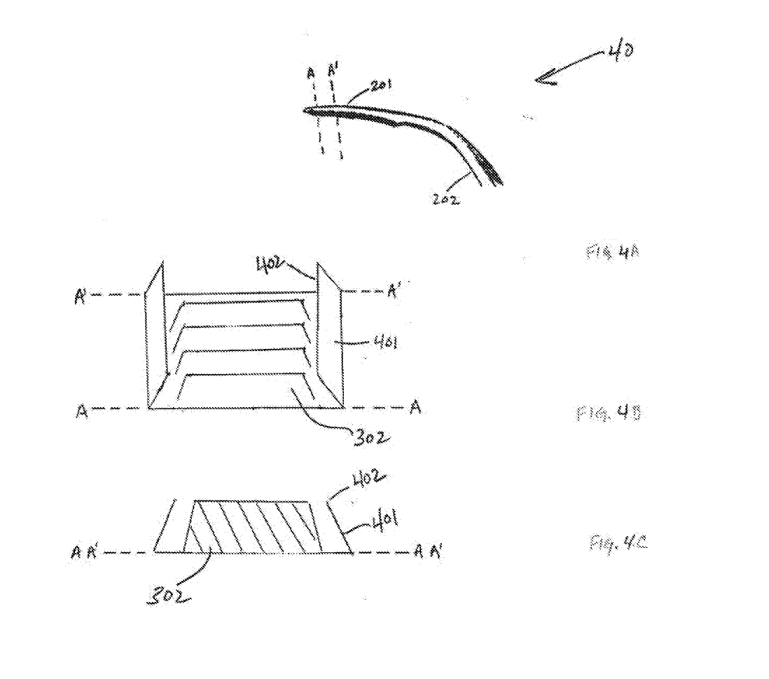

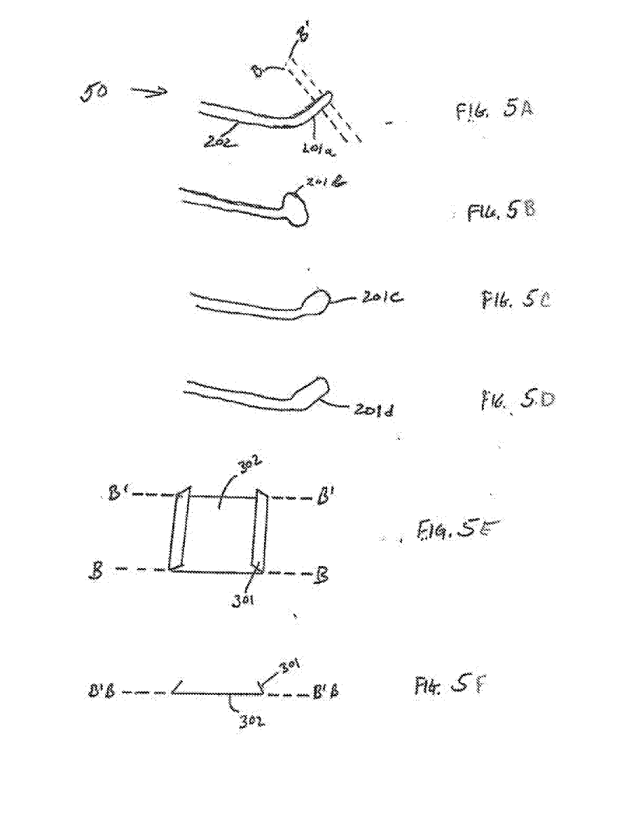

The cutting top edges of the teeth are most available for first contact with tissue when the instrument of the present invention is moved in the direction of the handle after insertion in a body where the angles formed by the proximal sides (i.e., toward the handle) of the teeth form an acute to right angle with respect to the longitudinal axis of the cutting element (see angles 303 and 304 in FIGS. 3B-F). More particularly, angles between the teeth proximal sides and the longitudinal axis of the cutting element potentiate contact with tissue when they are between about 10° and about 90°; between about 20° and about 80°; between about 30° and about 60°; between about 35° and about 55°; between about 40° and about 50°. In another embodiment, the angle formed by the proximal sides of the teeth and the longitudinal axis of the cutting element form an angle of about 10°, about 20°, about 30°, about 40°, about 45°, about 50°, about 60°, about 70°, about 80°, or about 90°. In addition to the alternative teeth designs shown in FIGS. 3A-F, FIGS. 2C-D show further alternative teeth designs shown in the context of the cutting element embodiment shown in FIGS. 2A and 2B.

The surgical instrument is nonetheless operative when the angles formed by the proximal sides of the teeth are obtuse with respect to the longitudinal axis of the cutting element (see angles 305 in FIG. 3A, for example). The instrument operated with suitable lateral pressure against target tissue intended to be removed will cut and remove the tissue, but at a slower rate than will the instruments just described having acute to right angles at the same location. There are scenarios where such a lesser efficient cutting instrument is the proper choice, providing the surgeon with added control when operating on tissue that is, for example, already compromised due to osteoporosis or other bone deficit malady.

In contrast to the more efficient cutting designs employed on the proximal side of the teeth, the determined angles formed between the distal ends (i.e., away from the handle) of the teeth and the longitudinal axis of the cutting element are obtuse (see angles 306a-d in FIGS. 3B-F). More particularly, the angle of the distal sides of the teeth serve to minimize the cutting ability of the teeth when moved forward or distally into a body. The distal side angle also affects spacing between the teeth. Accordingly, the cutting ability lessens and the spacing increases the more obtuse the distal side angle between tooth and longitudinal axis. Overall, distal angles range from about 95° and about 170°; between about 105° and about 160°; between about 115° and about 150°; between about 125° and about 145°; between about 130° and about 140°. In another embodiment, the angle formed by the distal sides of teeth and the longitudinal axis is about 95°, about 105°, about 115°, about 120°, about 125°, about 135°, about 145°, about 155°, about 165°, or about 170°.

A number of examples of different embodiments of the present invention featuring variant tooth designs is illustrated in FIGS. 3A-F. In these drawings, the stand-in structure 301 is used to indicate an additional length of shaft 202. The handle 203 in these illustrations is held substantially constant, leaving the focus on the cutting elements 205a-f. The teeth of cutting element 205a each describe regular equilateral triangles 302a; as such, angle 305 is 120°. As noted earlier, this design of cutting element maintains an ability to cut tissue when applied with suitable lateral force; and should do so equally well with forward or backward strokes. The surgical instrument of

FIG. 3B includes teeth that describe isosceles triangles with one of the equal sides in contact with the cutting side surface of the cutting element such that the proximal side describes acute angles 303 whereas the distal side describes obtuse angles 306a. FIG. 3C displays a surgical instrument whose teeth are right angles, where the proximal side describes a right angle 304 and the distal side describes an obtuse angle 306b. A fourth category of teeth that can be employed in the surgical instruments of the present invention is shown in FIG. 3E. Like tooth 302b, tooth 302d is essentially an isosceles triangle where the distal side has an obtuse angle 306d and the proximal side has an acute angle 303. The difference between tooth 302b and 302d is the portion at the top crown of the tooth that extends the distal edge beyond its proximal counterpart, thereby forming a razor-like added cutting edge oriented to cut tissue efficiently when the instrument is pulled back against target tissue.

One can readily manufacture surgical instruments that include combinations of differently configured teeth, as shown in FIGS. 3D and F. In FIG. 3D, right angle triangular teeth 302b are combined with the more aggressive category of the isosceles triangular teeth 302c. In FIG. 3F, the same right angle triangular teeth are combined with the far more aggressive isosceles triangle teeth 302d. The surgeon can use an instrument knowing that more or less aggressive teeth are located on different defined regions of the cutting element and use that knowledge to maintain control over the rate of cutting tissue, thus heightening the potential to avoid destabilizing the lumbar vertebrae when seeking to alleviate the painful symptoms of sciatica. The variations set forth in FIGS. 3A-F are merely exemplary; one skilled in the art can mix and match a plethora of tooth design for the cutting element, including the ordered rows of teeth as shown as well as less ordered configurations or randomly ordered configurations of such cutting edges. As noted by the stand-in length feature 301, the surgical instrument of the present invention can be literally any length desired. The bend of the arcuate element can dispose the instrument to be at any suitable angle usefully employed to address issues of placement of the instrument in a patient's body to be situated in a spinal canal 107 and continue from there into the neural foramen 110, which, in a nutshell, is the path of relief for a sciatica patient, as just one example where the present invention may be used.

The examples of various cutting element designs portrayed in FIGS. 3A-F do not present the convex base, concave cutting side, and smoothed, non-piercing distal tip that is portrayed in FIG. 2. Nonetheless, the various embodiments of tooth design can be readily employed on a cutting element that is curved or straight, as well as can the myriad other such embodiments that one skilled in the art can design.

The cutting element can include a second category of cutting surface, namely inclusion of a cutting edge at the perimeter of the cutting side of the cutting element. The cutting surface can be disposed at a right angle relative to the substantially planar surface of the cutting element. For example, as shown in FIGS. 4A-C, one can visualize the perimeter cutting edge by taking a slice of the cutting element 201 along lines A and A′. A top perspective view is shown in FIG. 4B, wherein the cutting element includes teeth 302 and a perimeter cutting edge 401. The cutting edge 401 is angled inward, forming an acute angle between the perimeter cutting surface and the cutting element surface. FIG. 4C illustrates the same slice of the cutting element, looking down the length of the section from one opened side. The perimeter cutting edge 402 and the cutting edge side 401 each show clearly the angle of the cutting edge and the beginning of the row of teeth. The design where the perimeter cutting edge is held between a right and an acute angle relative to the cutting element surface minimizes the potential to inadvertently inflict harm to the patient's tissue when inserting the instrument into the body or using it as a probe.

Yet another design of the surgical instrument of the present invention includes just the perimeter cutting edfe, as shown in FIGS. 5A-F. The shape of a cutting element employed in the context of the present invention can vary dramatically, including the following shapes that are offered without limitation intended: a substantially circular form 201c, a substantially oblong form 201b, a narrow trough-like form 201a, and a pancake flipper form 201d. A slice of the narrow trough through lines B and B′ serves to illustrate the basic form of these designs of the surgical instrument, as illustrated in FIGS. 5E (top perspective view) and 5F (side view), which demonstrate the same structures seen in FIGS. 4B and 4C, respectively, but without the centrally located teeth.

The surgical instruments of the present invention can be made from any material that affords sufficient integrity of form and flexibility to be capable of insertion into a body and laterally pressed against hard tissue while being drawn and pushed without breakage or splintering. Such a material must be non-toxic to a human when in contact with bodily fluids and tissues. Useful materials employed in the manufacture of surgical instruments of the present invention include stainless steel, titanium, and certain stiff polymers.

The method of manufacture of the surgical instrument described herein uses steps that are well-known to artisans of this field, and can vary per tolerances and manufacturing requirements of the equipment that one may use in such fabrication endeavors. In one manufacturing process, the elongate structure is formed from a suitable piece of stainless steel on a lathe, leaving a bulb at one end; the sides of the bulb then shaved off, the teeth ground in, and then the head that includes the teeth bent over.

The method of use of the surgical instrument of the present invention involves making an incision in the body of a patient proximate to tissue that is targeted to be removed from the patient. The surgical instrument of the present invention is particularly well-suited for removal of hard tissues, such as bone or ligaments. Any surgical procedure calling for removal of such tissue can be facilitated by careful use of the present invention particularly in view of its design for insertion into the body with minimal risk of damaging soft tissues while placing the instrument in contact with the targeted tissue for removal. In particular, it is contemplated that the present invention will find utility for surgeons performing bone or ligament reduction operations in the context of plastic surgery, ENT surgery, orthopedic surgery, and podiatric surgery, including in particular procedures directed at bone spurs and other bone deformities that may impact, for example, toe comfort and function, sciatic nerves, and carpal tunnel nerves.

In one procedure that usefully employs the instrument of the present invention, for relief of sciatica, the surgeon makes an incision in the body of a patient proximate to the lumbar vertebrae of interest, uncovering a surgical field that exposes the lumbar vertebrae, inserting the distal tip of the instrument into the spinal canal at a point that is adjacent to the intervertebral foramen from which the sciatic nerve root emerges from the spine, and then moving the surgical instrument forward such that the distal tip enters the intervertebral foramen such that the convex non-cutting side of the cutting element is in contact with nerve tissue and the concave cutting side of the cutting element is in contact with hard tissue surrounding the intervertebral foramen, the facet joint, and/or ligaments or other hard tissue that is in contact with the sciatic nerve root, and, carefully, removing some of the hard tissue that has narrowed the space occupied by the sciatic nerve root; following be removing the surgical instrument by moving it in reverse order to the above and closing the surgical field. Other procedures for removal of hard tissues follow a similar protocol in accordance with actions well-known to the field.

All technical terms have the standard accepted meaning in the art to which the present disclosure applies.

The above described methods of removing spinal blockages and the various surgical instruments described may be varied in many ways. In addition, a multiplicity of various features, both of methods and of instruments have been described. It should be appreciated that different features may be combined in different ways. In particular, not all the features shown above in a particular embodiment are necessary in every similar exemplary embodiment of the invention. Further, combinations of the above features are also considered to be within the scope of some exemplary embodiments of the invention. Also within the scope of the invention are surgical kits that include sets of surgical instruments suitable for relieving a stenosis and/or performing other parts of a surgical procedure.

When used in the following claims, the terms “comprise”, “include”, “have” and their conjugates mean “including but not limited to”.

As used herein, the singular form “a”, “an” and the include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly. the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from Ito 5, from 2 to 4, from 2 to 6, from 3 to 6 etc., as well as individual numbers within that range, for example, 1, 2, 3, 4, 5, or 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the modifier “about” before a numeral acts to include as equivalents additional numeral values as would be considered by one of ordinary skill in the art to be a trivial increase or decrease in numeral value. Depending on context as understood by the routineer, numeral values to be included are those that are as much as 50% plus or minus of the so-modified numeral.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

It will be appreciated by a person skilled in the art that the present invention is not limited by what has thus far been described. Rather, the scope of the present invention is limited only by the following claims and their equivalents.

What is claimed is:

1. A surgical instrument for removing tissue from a body, comprising an elongated shaft distally attached to a cutting element, wherein the elongated shaft and the cutting element are fixedly connected by an arcuate element such that the longitudinal axes of the elongated shaft and the cutting element intersect at an angle between about 90° and about 170° and the cutting element comprises a convex non-cutting base that contacts tissue when moving distally.

2. The surgical instrument of claim 1, wherein the arcuate element has a length substantially that of about a 30° to about a 60° arc of a circle having a radius that is between about 3.5 mm and about 6.5 mm.

3. The surgical instrument of claim 1, further comprising a handle proximally attached to the elongated shaft.

4. The surgical instrument of claim 1, wherein the surgical instrument comprises a metal or a polymer that is not toxic when employed in surgery.

5. The surgical instrument of claim 4, wherein the material is stainless steel.

6. The surgical instrument of claim 1, wherein the angle is between about 120° and about 150°.

7. The surgical instrument of claim 1, wherein the cutting element has a curvilinear distal end.

8. The surgical instrument of claim 7, wherein the curvilinear distal end is comprised of a convex side that is substantially free of a cutting surface and a concave side that comprises a cutting surface.

9. The surgical instrument of claim 1, wherein the cutting element's cross-sectional dimensions relative to its longitudinal axis have a length of between about 0.75 mm and about 5 mm.

10. The surgical instrument of claim 1, further comprising teeth that are disposed on a surface that is opposite the non-cutting base.

11. The surgical instrument of claim 10, wherein angles formed between proximal sides of teeth and the longitudinal axis of the cutting element form an angle between about 10° and about 90°.

12. The surgical instrument of claim 11, wherein the angles formed between proximal sides of teeth and the longitudinal axis are not all substantially the same.

13. The surgical instrument of claim 11, wherein the angles are substantially the same.

14. The surgical instrument of claim 10, further comprising a cutting edge disposed at the perimeter of the surface, wherein the surface and the cutting element contact each other at an angle of between about 90° and about 10°.

15. The surgical instrument of claim 1, wherein the cutting element has a surface opposite the non-cutting base and a cutting edge disposed at the perimeter of the surface, wherein the surface and the cutting element contact each other at an angle of between about 90° and about 10°.

16. The surgical instrument of claim 1, wherein length of the cutting element is between about 5 mm and about 18 mm.

17. The surgical instrument of claim 1, wherein the elongated shaft has a length range of about 10 cm to about 15 cm.

18. The surgical instrument of claim 1, wherein the elongated shaft tapers proximally to distally in its cross-section from about 1 cm to about 1 mm, respectively.

19. The surgical instrument of claim 18, wherein the elongated shaft tapers proximally to distally in its cross-section from about 7.5 mm to about 1.5 mm, respectively.

20. A method for removing targeted tissue from a body employing a surgical instrument that includes a distal cutting element that has a non-cutting convex side and a cutting concave side, comprising making a first incision in the body, passing the surgical instrument from outside the body to a point proximate to the targeted tissue, placing the surgical instrument's concave side of its distal end in contact with the targeted tissue, moving the surgical instrument back and forth along the longitudinal axis of the distal cutting element while applying pressure on the targeted tissue with the cutting concave side, and removing tissue that is freed from the body.
